(19) United States
(12) Patent Application Publication
(10) Pub. No.: US 2026/0138085 A1
(43) Pub. Date: May 21, 2026

(54) SYSTEMS AND METHODS FOR CARBON CAPTURE AND STORAGE

(71) Applicant: BluSky Carbon Inc., Old Saybrook, CT (US)

(72) Inventors: William Hessert, Ivoryton, CT (US); Hassan Khan, Old Saybrook, CT (US)

(21) Appl. No.: 19/392,652

(22) Filed: Nov. 18, 2025

Related U.S. Application Data
(60) Provisional application No. 63/722,429, filed on Nov. 19, 2024.

(51) Int. Cl. B01D 53/62; B01D 53/78; B01D 53/96; C01B 3/02; C01C 1/26; C07C 45/78; C10B 53/02; C10G 1/02

(52) U.S. Cl. CPC B01D 53/62; B01D 53/78; B01D 53/96; C01B 3/02; C01C 1/26; C07C 45/78; C10B 53/02; C10G 1/02; B01D 2252/102; B01D 2252/103; B01D 2257/504; B01D 2258/0283; C10G 2300/1011

(57) ABSTRACT

The present disclosure provides methods and systems for capturing carbon dioxide. An example system includes: a pyrolysis device configured to produce syngas and bio-oil from biomass; a power generator configured to produce electricity by combusting at least one of the syngas or the bio-oil; and a carbon capture system configured to use the electricity to capture carbon dioxide from a gas.

### CROSS-REFERENCE TO RELATED APPLICATIONS

This application claims the benefit of U.S. Provisional Patent Application No. 63/722,429, filed on Nov. 19, 2024, the entire contents of which are incorporated by reference herein.

### TECHNICAL FIELD

The present disclosure relates generally to the field of carbon dioxide removal and, more specifically, to methods and systems for hybrid, modular carbon capture and storage.

### BACKGROUND OF THE DISCLOSURE

Increased concentrations of greenhouse gasses in the atmosphere are driving massive changes in the Earth's climate. The most significant of these gasses is carbon dioxide (CO₂). Carbon dioxide removal (CDR) technology holds promise to directly reduce the concentration of carbon dioxide in the atmosphere, helping mitigate climate change.

Carbon mineralization, also known as carbon dioxide capture and storage by mineral carbonation (CCSM), holds promise for the permanent sequestration of carbon dioxide sourced from carbon dioxide removal or point-source carbon capture (PSC). Most focus has been on geological sequestration, or the pumping of carbon dioxide deep underground where it reacts with minerals to form stable carbonates. However, the lack of infrastructure, difficulty in permitting, and risk of either leakage or seismic activity, creates a need for a CCSM system that can be built above ground with verifiable, measurable, and rapid sequestration of carbon dioxide.

The foregoing examples of the related art and limitations therewith are intended to be illustrative and not exclusive, and are not admitted to be “prior art.” Other limitations of the related art will become apparent to those of skill in the art upon a reading of the specification and a study of the drawings.

### SUMMARY OF THE DISCLOSURE

The present disclosure addresses the above-mentioned problems and provides improved systems and methods for carbon dioxide removal, point-source carbon capture, and carbon storage or sequestration. In certain examples, the systems and methods can be deployed in areas where biomass sources are plentiful and/or access to external power sources (e.g., an electrical grid) is limited or unavailable. A pyrolysis process can be performed to convert biomass into syngas, bio-oil, and/or biochar. The syngas and/or bio-oil can be used to generate power or energy (e.g., electricity) for performing carbon capture and/or carbon storage processes. A variety of carbon capture and storage techniques can be utilized, as described herein.

In one aspect, the subject matter of this disclosure relates to a system for capturing carbon dioxide. The system includes: a pyrolysis device configured to produce syngas and bio-oil from biomass; a power generator configured to produce electricity by combusting at least one of the syngas or the bio-oil; and a carbon capture system configured to use the electricity to capture carbon dioxide from a gas (e.g., air or exhaust). In certain examples, the carbon capture system includes a first reactor configured to: receive the gas and a liquid including water and ammonia; and react the ammonia with the carbon dioxide to form an aqueous solution including ammonium bicarbonate (ABC) and/or ammonium carbonate (AC).

In some embodiments, the carbon capture system can include a second reactor configured to: combine the aqueous solution with at least one of a silicate or an oxide; and react the ABC and/or the AC with the at least one of the silicate or the oxide to form a carbonate and ammonia. The gas can be or include any gas that contains carbon dioxide, such as air or an exhaust from a combustion process. The at least one of the silicate or the oxide can be derived from or include at least one of industrial waste or a natural mineral. The at least one of the silicate or the oxide can include particles having an average particle size less than about 1 mm in diameter. The carbon capture system can include a filter for separating the carbonate from a liquid phase. The carbon capture system can include a tank for storing ammonia received from the second reactor.

In some implementations, the carbon capture system includes: a container configured to mix the aqueous solution including ABC and/or AC with acetaldehyde, wherein mixing the aqueous solution including ABC and/or AC with acetaldehyde releases carbon dioxide from the aqueous solution and forms a mixture including acetaldehyde and ammonia; a reactor configured to combine the mixture including acetaldehyde and ammonia with at least one of a silicate or an oxide to produce a carbonate and a liquid including acetaldehyde and ammonia; a filter for separating the carbonate from the liquid including acetaldehyde and ammonia; a calciner for heating the carbonate to release carbon dioxide and produce one or more of silicate or oxide; and a separator for receiving the liquid including acetaldehyde and ammonia and separating the acetaldehyde from the ammonia. The carbon capture system can further include: a first tank for storing ammonia received from the separator; and a second tank for storing acetaldehyde received from the separator. The separator can be a thermal separator. The carbon capture system can include at least one of a tank or a pipeline for receiving the carbon dioxide released in at least one of the container or the calciner. The carbon capture system can include a pump for pumping carbon dioxide from the tank or the pipeline into an underground formation.

In another aspect, the subject matter of this disclosure relates to a method of capturing carbon dioxide. The method includes: producing syngas and bio-oil from biomass in a pyrolysis device; producing electricity by combusting at least one of the syngas or the bio-oil in a power generator; and using the electricity to capture carbon dioxide from a gas in a carbon capture system. In certain examples, the method includes: receiving the gas and a liquid including water and ammonia; and reacting the ammonia with the carbon dioxide to form an aqueous solution including ammonium bicarbonate (ABC) and/or ammonium carbonate (AC).

In some implementations, the method includes: combining the aqueous solution with at least one of a silicate or an oxide; and reacting the ABC and/or the AC with the at least one of the silicate or the oxide to form a carbonate and ammonia. The at least one of the silicate or the oxide can be derived from or include at least one of industrial waste or a natural mineral. The method can include separating the carbonate from a liquid phase.

In certain examples, the method includes: mixing the aqueous solution including ABC and/or AC with acetaldehyde to release carbon dioxide from the aqueous solution and form a mixture including acetaldehyde and ammonia; combining the mixture including acetaldehyde and ammonia with at least one of a silicate or an oxide to produce a carbonate and a liquid including acetaldehyde and ammonia; separating the carbonate from the liquid including acetaldehyde and ammonia; heating the carbonate to release carbon dioxide and produce one or more of silicate or oxide; and separating the acetaldehyde from the ammonia in the liquid including acetaldehyde and ammonia.

The foregoing is a summary and thus contains, by necessity, simplifications, generalizations and omissions of detail; consequently, the summary is illustrative only and is not limiting in any way. Other aspects, inventive features, and advantages of the systems and/or processes described herein will become apparent in the non-limiting detailed description set forth herein.

### BRIEF DESCRIPTION OF THE DRAWINGS

These and other features, aspects, and advantages of the present disclosure will become better understood with regard to the following description, and accompanying drawings.

FIG. 1 is a schematic diagram of a system for capturing carbon dioxide from one or more gas sources, in accordance with certain embodiments.

FIG. 2 is a schematic diagram of a carbon capture system, in accordance with an embodiment.

FIG. 3 is a schematic diagram of a carbon capture system, in accordance with an embodiment.

In the drawings, like reference characters generally refer to the same parts throughout the different views. Also, the drawings are not necessarily to scale, emphasis instead generally being placed upon illustrating the principles of the invention.

### DETAILED DESCRIPTION

To make the aforementioned objects, features and advantages of the present disclosure more obvious and understandable, the present disclosure will be further described below with reference to the accompanying drawings and embodiments. It should be noted that specific details are set forth in the following description to fully understand the present disclosure. However, the present disclosure may be implemented in many other ways different from those described herein, and those skilled in the art may make similar generalizations without violating the connotation of the present disclosure. Therefore, the present disclosure is not limited by the specific embodiments disclosed below.

It should be noted that the example embodiments may be implemented in various forms, and should not be construed as being limited to the embodiments set forth herein. On the contrary, the provision of these embodiments makes the present disclosure more comprehensive and complete, and fully conveys the concept of the example embodiments to those skilled in the art. The same reference numerals in the figures indicate the same or similar structures, and thus their repeated description will be omitted. In addition, the similarities between the embodiments will not be repeated.

In various examples, “biomass” is organic matter derived from plants or animals. Biomass can include or be derived from, for example, trees, branches, shrubs, bushes, grasses, algae, seaweed, kelp, forestry residue, wood, sawdust, agricultural crops, agricultural waste, animal waste, municipal waste, or any combination thereof.

In the past years, global CO₂ concentrations have increased from 280 parts per million to almost 420 parts per million. To mitigate the effects of climate change and keep global warming to 1.5° C. above pre-industrial levels, the United Nations Intergovernmental Panel on Climate Change (IPCC) laid out a plan to bring humanity to net zero emissions by 2050. Experts from many organizations, including the IPCC, claim that gigatonne-scale CDR technologies will be required to reach net zero goals, potentially up to 13 billion tonnes of CDR per year. However, the cost of CDR has remained higher than hoped largely due to the intensive energy requirements of existing CDR processes. To make CDR financially feasible, costs may be required to drop below $100 per tonne of carbon dioxide. This would place the costs of CDR in line with other waste products and would make the technology economically competitive.

The methods and systems disclosed herein enable this combined goal of scale and cost to be achieved through use of a hybrid CDR system that, in various examples, combines technologies that can be mass-produced and operated for little cost. In some instances, for example, the systems disclosed herein can be integrated into existing CDR and CCS systems and/or can use industrial waste to directly mineralize carbon dioxide into one or more carbonates, such as calcium carbonate and/or magnesium carbonate. The process can take place at temperatures much lower than those required for carbon dioxide removal in other CDR and CCS systems. Further, in some examples, silicate and/or oxide minerals in the industrial waste can be crushed into small particles, to increase solubility in water and/or an available surface area for reaction with carbon dioxide. Such techniques can make the systems and methods described herein more efficient and scalable.

### System Overview

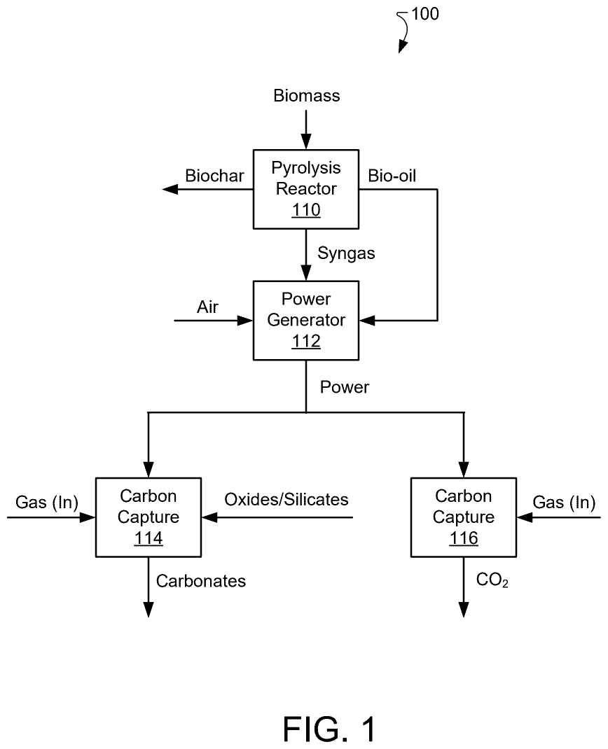

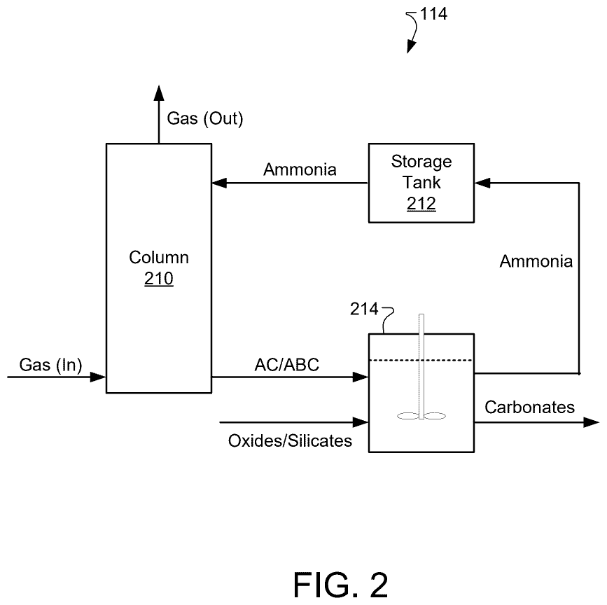

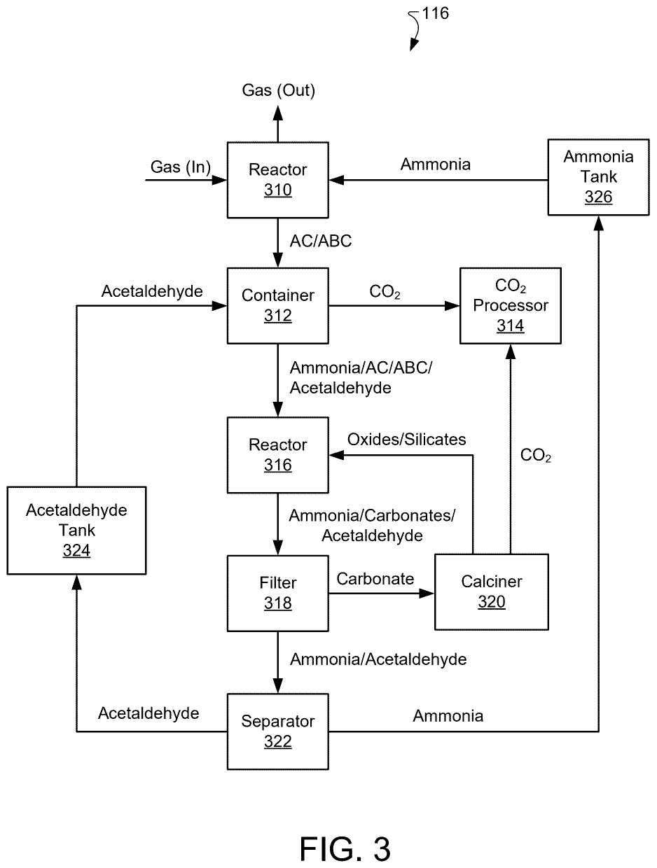

FIG. 1 is a schematic diagram of a system 100 for capturing carbon dioxide from one or more gas sources (e.g., air or exhaust), in accordance with certain examples. The system includes a pyrolysis device 110 that converts biomass into biochar, bio-oil, and syngas. The syngas and/or the bio-oil can be combusted in a power generator 112 that generates electricity and/or other form of power or energy (e.g., mechanical energy or thermal energy). The electricity and/or other power or energy can be provided to a carbon capture system 114 that captures carbon dioxide from air or other gas (e.g., exhaust from a combustion process) and reacts the carbon dioxide with one or more oxides and/or silicates to form one or more carbonates. The oxides and/or silicates can be obtained from a waste product, such as waste concrete, or a natural mineral, as described herein. The electrical power can also be provided to a carbon capture system 116 that captures carbon dioxide from air or other gas (e.g., exhaust) and outputs a stream of carbon dioxide (e.g., in gaseous form). The carbon capture system 114 and the carbon capture system 116 are described in more detail with reference to FIGS. 2 and 3, respectively. In various examples, the carbon capture system 114 and/or the carbon capture system 116 can be used for either direct air capture (e.g., capturing carbon dioxide from air) or point source capture (e.g., capturing carbon dioxide from exhaust).

Still referring to FIG. 1, in various examples, the biological carbon cycle naturally grows biomass by absorbing atmospheric carbon dioxide through photosynthesis. When the biomass dies and decomposes or burns, carbon from the biomass can be returned to the atmosphere as carbon dioxide. Preventing this release of biological carbon into the atmosphere is carbon-negative and/or achieves CDR.

Pyrolysis is one CDR approach that can prevent the release of biological carbon into the atmosphere as carbon dioxide. For example, the pyrolysis device 110 can heat the biomass in a low-oxygen environment at high temperatures (e.g., 350-900° C.), to thermally decompose the biomass into biochar, bio-oil, and syngas. In various examples, biochar can include stable aromatic carbon that locks carbon (e.g., in soil) for hundreds or thousands of years. The biochar produced by the pyrolysis device 110 can be deposited on fields for use as fertilizer or soil amendments, put in lagoons (e.g., with pig waste) to contain or stabilize contaminants, and/or stored in a landfill, quarry, or other location.

Generally, higher pyrolysis temperatures can produce biochar of sufficient quality to make the biological carbon resistant to degradation on geological timescales (millions of years); however, such temperatures can decrease biochar yield and/or reduce carbon efficiency of the pyrolysis system. To address these limitations, the bio-oil and syngas generated from the pyrolysis device 110 can be converted into or used to produce sequestered forms of carbon. This can be done, for example, with geological sequestration of carbon dioxide using a carbon capture and storage (CCS) system, where carbon dioxide from exhaust gasses (e.g., from combustion of the syngas and/or the bio-oil) is captured and further processed, as described herein.

In various examples, syngas is a flammable gas containing hydrogen and carbon monoxide that can be used to provide thermal or electrical energy. For example, the power generator 112 may utilize syngas to power the pyrolysis device 110 and/or related components, making the pyrolysis process self-sustaining.

By comparison, the bio-oil may be more difficult to combust and/or may need to be upgraded to reduce an oxygen content and increase a heating value. For example, the bio-oil may need to be dewatered. Additionally or alternatively, the bio-oil can be distilled to create light hydrocarbons, such as propane and/or methane, which can be stored onsite and/or used to jumpstart the pyrolysis process. In some examples, the bio-oil can be gasified to produce syngas (e.g., containing hydrogen and/or carbon monoxide), which can be combusted in the power generator 112.

Still referring to FIG. 1, in various examples, the electrical power from the power generator 112 can be used to drive processes or operations taking place in or around the carbon capture system 114 and/or the carbon capture system 116. For example, the electrical power can be used to operate pumps, conveyors, mixers, crushing devices (e.g., for crushing industrial waste, silicates, and/or oxides into small particles), filters, and/or other equipment used to capture carbon dioxide (e.g., from air or exhaust produced from combusted syngas and/or bio-oil) and produce carbonates in the carbon capture system 114. Additionally or alternatively, the electrical power can be used to operate equipment (e.g., large fans, pumps, etc.) for capturing carbon dioxide (e.g., from air or exhaust) in the carbon capture system 116. In another example, surplus electricity from the electrical power can create baseload renewable power for a local grid.

In various examples, the pyrolysis reactor 110, the power generator 112, the carbon capture system 114, the carbon capture system 116, or any combination thereof can be located in close proximity to one another (e.g., on a common parcel of land), can be in fluid communication with one another, can exchange materials with one another, can exchange power or energy with one another, and/or may or may not be physically attached to one another. For example, the power generator 112 can be wired directly to or can be in fluid communication with the carbon capture system 114 and/or the carbon capture system 116. This can allow the power generator 112 to provide some or all of the electrical power and/or thermal energy needed to operate the carbon capture system 114 and/or the carbon capture system 116. Advantageously, this can allow the system 100 to be located and operated in areas where there is no access to external power sources. For example, the system 100 can be located in remote areas where biomass sources are plentiful (e.g., in or near forests or farmland) and/or where there is no electrical power grid, natural gas, or other external power sources.

### Carbon Capture Using Mineralization

FIG. 2 is a schematic diagram of the carbon capture system 114, in accordance with certain embodiments. The carbon capture system 114 includes two main processes: (1) an absorption process for absorbing carbon dioxide from a gas (e.g., air and/or an exhaust gas from combustion of bio-oil and/or syngas) using a solution including water and ammonia (alternatively referred to as “ammonium hydroxide” or “aqueous ammonia”), to form ammonium bicarbonate (NH₄HCO₃ or “ABC”) and/or ammonium carbonate ((NH₄)₂CO₃ or “AC”); and (2) a carbonation process for reacting one or more silicates and/or oxides with ABC and/or AC to form one or more carbonates.

In various examples, the absorption process for absorbing carbon dioxide can be performed in a reactor for liquid/gas reactions, such as a packed column 210. The column 210 can receive a gas flow at a bottom portion of the column 210 and a liquid flow at a top portion of the column 210. As the gas flows upward and the liquid flows downward through the column 210, carbon dioxide in the gas can be absorbed by the liquid. The column can include one or more sections of randomized packing or structured packing designed to maximize a surface area for gas-liquid contact. In one example, 10 mm pall rings are used as the packing material. The packing material can be held in place with one or more supports and/or restrainers. In some examples, the absorption process can take place at a temperature from about 5° C. to about 10° C. The gas and/or liquid entering the column 210 can be cooled and/or heated to achieve a desired temperature.

A pump can be used to transport aqueous ammonia from a storage tank 212 to the top portion of the column 210, where a distributor (e.g., including one or more spray nozzles) can dispense the aqueous ammonia onto the packing material. One or more pH sensors can be included to monitor and/or control a pH of the aqueous ammonia for achieving efficient CO₂ absorption. One or more additional sensors can be used to monitor concentrations of CO₂ entering and exiting the column 210. The resultant solution containing AC and/or ABC (alternatively referred to as “AC/ABC solution”) exiting the column 210 can be stored in a storage tank prior to being delivered to the carbonation process to form carbonates, as described herein.

In some embodiments, some or all portions of the column 210, packing, piping, etc., can be constructed of corrosion-resistant materials, such as 304 or 316 Stainless Steel, to prevent corrosion caused by alkaline ammonium hydroxide. Likewise, storage tanks can be built or lined with polypropylene to avoid corrosion. Other corrosion-resistant materials can be used, for example, depending on a composition and/or pH of the liquid.

With previous carbon capture approaches, carbon dioxide is thermally stripped from an AC/ABC solution to produce carbon dioxide for geological sequestration and to regenerate the absorbent solvent. By comparison, use of the carbonation process described herein can significantly reduce infrastructure and energy requirements. In some instances, for example, the carbonation process can be performed using oxides and/or silicates derived from industrial waste.

Many industrial wastes suitable for use in the systems and methods described herein (e.g., waste concrete, fly ash, red must, ferrous slags, and more) have high concentrations of reactive silicates (e.g., calcium silicates), calcium oxide (CaO), and/or magnesium oxide (MgO). These silicates and oxides can react with carbon dioxide to form carbonates that are stable on geological timescales. For example, CaO and MgO can react with carbon dioxide to form carbonates, as follows:

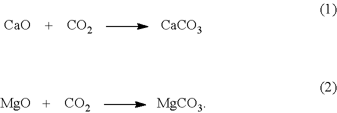

While these silicates and oxides are reactive, they are often trapped inside a solid material and/or not exposed to carbon dioxide, such that carbonation reactions are unable to take place. For instance, in a piece of waste concrete, carbonate reactions may occur only on outer surfaces of the concrete, where silicates and/or oxides can be exposed to carbon dioxide. To increase surface area and overall exposure of the silicates and/or oxides to carbon dioxide, the industrial waste and/or other sources of silicates and/or oxides described herein can be milled, crushed, and/or pulverized into small particle sizes. The particles can have an average or maximum size (e.g., diameter) that is less than or equal to about, for example, 1 mm, 2 mm, or 5 mm.

In various examples, the carbonation process can take place in a reactor 214, such as a stirred tank reactor or a jacketed stirred reactor, where particles containing silicates and/or oxides are mixed with the AC/ABC solution. This carbonates the reactive silicates and/or oxides and regenerates the aqueous ammonia solution. The reactions can be completed over the course of minutes to hours. The reactions involving CaO and MgO can be as follows:

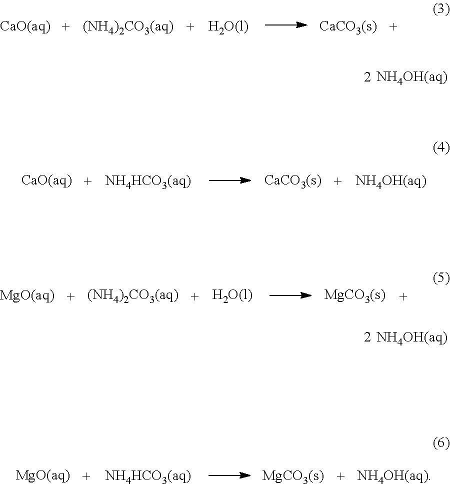

In general, calcium and magnesium oxides dissolve in water, as does AC, ABC, and ammonia; however, calcium and magnesium carbonate are highly insoluble in water, especially in alkaline environments (which the ammonia creates). This allows the carbonates to precipitate out of the solution and be physically filtered out. For example, once the reaction has been completed, the contents from the reactor 214 can be passed through a filter to separate particles of solid carbonate from liquid components.

The carbonates produced in the reactor 214 can be used as filler materials or aggregates for building products (e.g., concrete) and/or can be sequestered in an old mine, quarry, landfill, or other location. Other uses for the carbonates are contemplated.

The liquid components exiting the reactor 214 can be or include an ammonia solution, which can be stored in the storage tank 212. Any oxides and/or silicates present in the ammonia solution can assist with absorption of carbon dioxide when the ammonia solution is recycled back to the column 210.

In some embodiments, pretreatment or post treatment of industrial waste streams can be performed, for example, before the waste streams are added to the reactor 214. Such pretreatment and/or post treatment processes can include, for example, grinding (e.g., to reduce particle sizes and increase surface area) and/or thermal activation (e.g., to increase reactivity).

In some embodiments, silicates used in the reactor 214 can be obtained from natural minerals, such as olivine, serpentine, and/or wollastonite. Table 1 lists example minerals along with their chemical formulas and a description of their abundance on Earth.

TABLE 1
Examples of silicate sources.

| Mineral | Chemical Formula | Abundance on Earth |
|---|---|---|
| Olivine | Mg₂SiO₄ | >50% of the Earth's upper mantle |
| Serpentine | Mg₃Si₂O₅(OH)₄ | Considered widely abundant on Earth |
| Wollastonite | CaSiO₃ | 100 million tonnes |

In certain examples, one advantage of the carbonation process disclosed herein is that it eliminates the need to thermally strip carbon dioxide from a solvent, which is one of the largest costs and energy requirements of traditional carbon capture technologies. In addition, the disclosed process can eliminate or minimize the need for complex carbon sequestration infrastructure, such as pipelines and wells used in existing carbon removal systems.

In some embodiments, alkaline materials (e.g., from waste concrete) can be used to passively absorb carbon dioxide from the atmosphere, for example, through enhanced rock weathering. The alkaline materials can be mixed with biochar and/or can be spread over a field or other surface. Alkaline materials for such purposes can include or be derived from, for example, waste concrete, olivine, serpentine, or other sources. The alkaline materials can be ground into small particles to increase surface area.

In various examples, gas fed into the column 210 can be any type of gas that includes carbon dioxide. The gas can be or include, for example, air, an exhaust from combustion of syngas and/or bio-oil, an exhaust produced by a coal plant, gas turbine, bioenergy facility, train/locomotive, and/or other exhaust source.

### Carbon Capture and Sequestration

In various examples, the systems and methods described herein can be used to capture carbon dioxide directly from air (e.g., using the carbon capture system 116). One issue with direct air capture is high energy requirements. By the year 2100, direct air capture facilities may use up to a quarter of global energy, unless substantial energy improvements are made. A large portion of the energy consumed in existing direct air capture processes goes into stripping carbon dioxide from the solvent. In general, the better a solvent is at absorbing atmospheric carbon dioxide, the more difficult it is to separate the carbon dioxide from the solvent. The carbonation process described herein may overcome this limitation by carbonating alkaline materials (e.g., having a pH from about 8, to about 9, to about 10, to about 11, to about 12, or higher). At the same time, surplus energy created by combusting syngas and/or bio-oil (e.g., using the power generator 112) can be directed to powering large fans for direct air capture, crushing alkaline materials into small particles, and other electrical requirements, as described herein.

FIG. 3 is a schematic diagram of the carbon capture system 116, in accordance with certain embodiments. The carbon capture system 116 captures carbon dioxide from a gas (e.g., air, exhaust, or any other gas containing carbon dioxide), for example, by drawing large volumes of the gas into a reactor 310, which can be or include a packed column (similar or identical to the column 210). The gas can be drawn into the reactor 310 using fans powered by the power generator 112 or other source. An aqueous ammonia solution can be added to the reactor 310 such that carbon dioxide in the gas can be absorbed by the aqueous ammonia solution to form a solution containing AC and/or ABC, as described herein. A gas containing less carbon dioxide than the input gas can be removed from the reactor 310. In various examples, the reactor 310 can be or include a column in which a gas phase (e.g., air) enters a bottom portion of the column and exits from a top portion of the column, and a liquid phase enters from the top portion and exits from the bottom portion (similar to the flow in the column 210).

The AC/ABC solution can then be mixed in a container 312 with acetaldehyde or a similar material that reduces a solubility of carbon dioxide in the solution. This can liberate carbon dioxide, which can be provided to a CO₂ processor 314, where the carbon dioxide can be stored, transported, added to a pipeline, and/or injected underground for geological sequestration (e.g., using a pump). Other uses for the carbon dioxide are contemplated. Releasing the carbon dioxide in the container 312 can convert some or most of the AC and/or ABC to ammonia, such that the remaining aqueous solution can include ammonia, AC, ABC, acetaldehyde, or any combination thereof.

Next, the solution exiting the container 312 can be provided to a reactor 316 (e.g., a stirred tank reactor, such as the reactor 214) and mixed with one or more silicates and/or oxides, which react with the AC and/or ABC to form one or more carbonates (e.g., according to reactions (3) to (6)). The oxide(s) can include, for example, CaO, MgO, and/or other oxides, and can be in particulate form, as described herein. Additionally or alternatively, the silicates and/or oxide(s) can be derived from one or more waste products or natural minerals, as described herein. The materials exiting the reactor 316 can include the carbonate(s) and an aqueous solution containing ammonia and acetaldehyde. The carbonate(s) can be separated from liquid components using a filter 318. The filtered carbonate(s) can be provided to a heater or calciner 320, which can thermally strip carbon dioxide from the carbonate(s) (e.g., at a temperature from about 800° C., to about 900° C., to about 1000° C., or higher) to produce carbon dioxide gas and silicate(s) and/or oxide(s). The carbon dioxide can be provided to the CO₂ processor 314 for storage, transport, and/or sequestration, as described herein. The silicate(s) and/or oxide(s) can be stored and/or recycled to the reactor 316.

The filtrate exiting the filter 318 can be provided to a separator 322, which separates the acetaldehyde from the ammonia. In some instances, for example, the separator 322 can heat the filtrate and/or condense evaporated gases to achieve the separation. For example, the acetaldehyde can be separated from the ammonia by heating the filtrate to a temperature from about 60° C., to about 70° C., to about 80° C., or higher. The acetaldehyde can be provided to an acetaldehyde tank 324 for storage. Likewise, the ammonia can be provided to an ammonia tank 326 for storage (e.g., as aqueous ammonia).

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention.

The construction and arrangement of the elements of the apparatus as shown in the exemplary embodiments is illustrative only. Although only a certain number of embodiments have been described in detail in this disclosure, those skilled in the art who review this disclosure will readily appreciate that many modifications are possible (e.g., variations in sizes, dimensions, structures, shapes, and proportions of the various elements, values of parameters, mounting arrangements, use of materials, colors, orientations, etc.) without materially departing from the novel teachings and advantages of the subject matter recited.

Further, elements shown as integrally formed may be constructed of multiple parts or elements shown as multiple parts may be integrally formed, the operation of the assemblies may be reversed or otherwise varied, the length or width of the structures and/or members or connectors or other elements of the system may be varied, the nature or number of adjustment or attachment positions provided between the elements may be varied. It should be noted that the elements and/or assemblies of the system may be constructed from any of a wide variety of materials that provide sufficient strength or durability. Accordingly, all such modifications are intended to be included within the scope of the present disclosure. Other substitutions, modifications, changes, and omissions may be made in the design, operating conditions, and arrangement of the exemplary embodiments without departing from the spirit of the present subject matter.

The features and functions of the various embodiments may be arranged in various combinations and permutations, and all are considered to be within the scope of the disclosed invention. Accordingly, the described embodiments are to be considered in all respects as only illustrative and not restrictive. Furthermore, the configurations, materials, and dimensions described herein are intended as illustrative and in no way limiting. Similarly, although physical explanations have been provided for explanatory purposes, there is no intent to be bound by any particular theory or mechanism, or to limit the claims in accordance therewith.

It should be also understood that as used in the description herein the meaning of “a,” “an,” and “the” includes plural reference unless the context clearly dictates otherwise. Also, as used in the description herein, the meaning of “in” includes “in” and “on” unless the context clearly dictates otherwise.

Each numerical value presented herein is contemplated to represent a minimum value or a maximum value in a range for a corresponding parameter. Measurements, sizes, amounts, and the like may be presented herein in a range format. The description in range format is provided merely for convenience and brevity and should not be construed as an inflexible limitation on the scope of the invention. Accordingly, the description of a range should be considered to have specifically disclosed all the possible subranges as well as individual numerical values within that range. For example, description of a range such as 1-20 meters should be considered to have specifically disclosed subranges such as 1 meter, 2 meters, 1-2 meters, less than 2 meters, 10-11 meters, 10-12 meters, 10-13 meters, 10-14 meters, 11-12 meters, 11-13 meters, etc.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention.

Reference in the specification to “one embodiment,” “preferred embodiment,” “an embodiment,” “some embodiments,” or “embodiments” means that a particular feature, structure, characteristic, or function described in connection with the embodiment is included in at least one embodiment of the invention and may be in more than one embodiment. Also, the appearance of the above-noted phrases in various places in the specification is not necessarily referring to the same embodiment or embodiments.

What is claimed is:

1. A system for capturing carbon dioxide, the system comprising:
a pyrolysis device configured to produce syngas and bio-oil from biomass;
a power generator configured to produce electricity by combusting at least one of the syngas or the bio-oil; and
a carbon capture system configured to use the electricity to capture carbon dioxide from a gas.

2. The system of claim 1, wherein the carbon capture system comprises a first reactor configured to:
receive the gas and a liquid comprising water and ammonia; and
react the ammonia with the carbon dioxide to form an aqueous solution comprising ammonium bicarbonate (ABC) and ammonium carbonate (AC).

3. The system of claim 2, wherein the carbon capture system further comprises a second reactor configured to:
combine the aqueous solution with at least one of a silicate or an oxide; and
react the ABC and the AC with the at least one of the silicate or the oxide to form a carbonate and ammonia.

4. The system of claim 3, wherein the gas includes an exhaust from a combustion process.

5. The system of claim 3, wherein the at least one of the silicate or the oxide is derived from at least one of industrial waste or a natural mineral.

6. The system of claim 3, wherein the at least one of the silicate or the oxide comprises particles having an average particle size less than about 1 mm in diameter.

7. The system of claim 3, wherein the carbon capture system further comprises a filter for separating the carbonate from a liquid phase.

8. The system of claim 3, wherein the carbon capture system further comprises a tank for storing ammonia received from the second reactor.

9. The system of claim 2, wherein the carbon capture system comprises:
a container configured to mix the aqueous solution comprising ABC and AC with acetaldehyde, wherein mixing the aqueous solution comprising ABC and AC with acetaldehyde releases carbon dioxide from the aqueous solution and forms a mixture comprising acetaldehyde and ammonia;
a reactor configured to combine the mixture comprising acetaldehyde and ammonia with at least one of a silicate or an oxide to produce a carbonate and a liquid comprising acetaldehyde and ammonia;
a filter for separating the carbonate from the liquid comprising acetaldehyde and ammonia;
a calciner for heating the carbonate to release carbon dioxide and produce one or more of silicate or oxide; and
a separator for receiving the liquid comprising acetaldehyde and ammonia and separating the acetaldehyde from the ammonia.

10. The system of claim 9, wherein the gas is air.

11. The system of claim 9, wherein the carbon capture system further comprises:
a first tank for storing ammonia received from the separator; and
a second tank for storing acetaldehyde received from the separator.

12. The system of claim 9, wherein the separator is a thermal separator.

13. The system of claim 9, wherein the carbon capture system further comprises at least one of a tank or a pipeline for receiving the carbon dioxide released in at least one of the container or the calciner.

14. The system of claim 13, further comprising a pump for pumping carbon dioxide from the tank or the pipeline into an underground formation.

15. A method of capturing carbon dioxide, the method comprising:
producing syngas and bio-oil from biomass in a pyrolysis device;
producing electricity by combusting at least one of the syngas or the bio-oil in a power generator; and
using the electricity to capture carbon dioxide from a gas in a carbon capture system.

16. The method of claim 15, further comprising:
receiving the gas and a liquid comprising water and ammonia; and
reacting the ammonia with the carbon dioxide to form an aqueous solution comprising ammonium bicarbonate (ABC) and ammonium carbonate (AC).

17. The method of claim 16, further comprising:
combining the aqueous solution with at least one of a silicate or an oxide; and
reacting the ABC and the AC with the at least one of the silicate or the oxide to form a carbonate and ammonia.

18. The method of claim 17, wherein the at least one of the silicate or the oxide is derived from at least one of industrial waste or a natural mineral.

19. The method of claim 17, further comprising separating the carbonate from a liquid phase.

20. The method of claim 16, further comprising:
mixing the aqueous solution comprising ABC and AC with acetaldehyde to release carbon dioxide from the aqueous solution and form a mixture comprising acetaldehyde and ammonia;
combining the mixture comprising acetaldehyde and ammonia with at least one of a silicate or an oxide to produce a carbonate and a liquid comprising acetaldehyde and ammonia;
separating the carbonate from the liquid comprising acetaldehyde and ammonia;
heating the carbonate to release carbon dioxide and produce one or more of silicate or oxide; and
separating the acetaldehyde from the ammonia in the liquid comprising acetaldehyde and ammonia.
